(19) United States
(12) Patent Application Publication
(10) Pub. No.: US 2014/0105875 A1
(43) Pub. Date: Apr. 17, 2014

(54) NUTRITIONAL COMPOSITIONS HAVING EXOGENOUS MILK FAT GLOBULE MEMBRANE COMPONENTS

(71) Applicants: Doug Bolster, Lake in the Hills, IL (US); Zamzam (Fariba) Roughead, Plymouth, MN (US)

(72) Inventors: Doug Bolster, Lake in the Hills, IL (US); Zamzam (Fariba) Roughead, Plymouth, MN (US)

(73) Assignee: NESTEC S.A., Vevey (CH)

(21) Appl. No.: 14/123,700

(22) PCT Filed: Jun. 8, 2011
(86) PCT No.: PCT/US2011/039612

§ 371 Date: Dec. 3, 2013

(51) Int. Cl. A61K 35/20; A23L 1/305; A23L 1/30; A61K 35/74

(52) U.S. Cl. CPC A61K 35/20; A61K 35/745; A61K 35/747; A23L 1/3056; A23L 1/3014
USPC 424/93.45; 424/535; 424/93.4

(57) ABSTRACT

Nutritional compositions including milk fat globule membrane components (“MFGM”) and at least one nutrient are provided. The milk-derived, bioactive lipids help to modulate conditions typically found in the elderly including, for example, low-grade inflammation, loss of lean body mass, skeletal muscle cell membrane instability, and joint inflammation. The nutrients may include, but are not limited to, whey protein micelles, citrulline, branched chain fatty acids, and a-hydroxycaproic acid (“α-HICA”). Methods for treating an individual having, or at risk of having, a medical condition are also provided.

### BACKGROUND

There are many types of nutritional compositions currently on the market. Nutritional compositions can be targeted toward certain consumer types, for example, young, elderly, athletic, etc., based on the specific ingredients of the nutritional composition. Nutritional compositions can also be formulated based on the certain physiological conditions that the nutritional compositions are intended to treat or improve, or may be based on desired physical or organoleptic properties of the nutritional compositions.

One goal of nutritional support is to provide specific types and amounts of nutrients in nutritional compositions to provide a consumer with a sufficient amount of the nutrient to achieve a desired biological result. For example, the elderly and individuals with certain illnesses can often times experience low grade inflammation, skeletal muscle cell membrane instability, and a reduction in lean body mass that is due, at least in part, to a reduction in muscle protein synthesis. A reduction in lean body mass can lead to the loss of independence, functionality, and quality of life, as well as insulin sensitivity/glucose tolerance. Nutritional compositions can be formulated to treat or prevent such conditions.

However, many nutrients that are used in nutritional compositions to combat these types of aging- and/or illness-related conditions impart an undesirable taste or odor to the composition making it unappealing for consumption. As a result, the desired biological result is not achieved when the consumer refuses to ingest the composition due to its poor organoleptic properties. Thus, it is desired to provide nutritional compositions having specific types and amounts of nutrients that combat the effects of aging, while at the same time providing tolerable physical and organoleptic properties.

### SUMMARY

The present disclosure relates to nutritional compositions having milk fat globule membranes components (“MFGM”) and at least one nutrient. The present disclosure also relates to nutritional compositions for treating a range of physio-pathological conditions such as low-grade inflammation, loss of lean body mass, skeletal muscle cell membrane instability, joint inflammation, poor bone health, or combinations thereof. The nutritional compositions are particularly well-suited for use in the elderly, or individuals in need of treatment for the above-mentioned conditions. The skilled artisan will appreciate, however, that the present nutritional compositions may provide benefits to individuals having conditions not expressly mentioned herein.

In an embodiment, a nutritional composition is provided and includes milk fat globule membrane (“MFGM”) and at least one nutrient. The nutrient may be selected from the group consisting of whey protein micelles, alpha-hyroxyisocaproic acid (“α-HICA”), citrulline, branched chain fatty acids, or combinations thereof.

In an embodiment, the whey protein micelles are a source of at least one branched chain amino acid selected from the group consisting of leucine, isoleucine, valine, or combinations thereof.

In an embodiment, the nutritional composition further includes a probiotic selected from the group consisting of Bifidobacterium lactis CNCM I-3446, Lactobacillus rhamnosus GG ATCC 53103, Lactobacillus rhamnosus CGMCC 1.3724, Bifidobacterium longum BB536 deposited under ATCC BAA-999, Lactobacillus Reuteri ATCC55730, Lactobacillus Reuteri DSM-17938, Lactobacillus paracasei CNCM I-2116, Lactobacillus johnsonii CNCM I-1225, Lactobacillus helveticus CNCM I-4095, Bifidobacterium breve CNCM I-3865, Bifidobacterium longum CNCM I-2618, or combinations thereof. The MFGM may include proteins or bioactive proteins able to bind with or biologically interact with the probiotic.

In an embodiment, the MFGM includes gangliosides or phospholipids able to bind with or biologically interact with the probiotic, the gangliosides or phospholipids being present in an amount between 0.03% and 5% by weight of total proteins.

In an embodiment, the MFGM is present in an amount of between 0.1% and 15% by weight of total proteins. The MFGM may also be present in an amount between 0.01 g and 15 g of MFGM per 100 g of the nutritional composition.

In an embodiment, the nutritional composition further includes a prebiotic selected from the group consisting of fructo-oligosaccharides, galacto-oligosaccharides, cow milk oligosaccharides, or combinations thereof.

In an embodiment, the MFGM originates from a source selected from the group consisting of butter milk, butter milk fractions, defatted butter milk, delactosylated buttermilk, buttermilk fraction obtained by microfiltration or ultrafiltration, fractions recovered from whey protein concentrate, sweet whey, acid whey, whey cream or fat associated fraction from whey containing phospholipids, or combinations thereof.

In an embodiment, the MFGM includes a component selected from the group consisting of sphingomyelin, phosphatidyl ethanolamine, phosphatidylcholine, phosphatidyl inositol, phosphatidyl serine, cholesterol, gangliosides, mucin1, xantine-oxidase/dehydrogenase, periodicacid schiff, CD36, butyrophilin, adipophilin, PAS 6/7, fatty-acid binding protein, lactoferrin, lactaldherin, peptide ETTVFENLPEK, peptide SFQLFGSPPGQR, peptide GSNFQLDQLQGR, peptide FQFIQVAGR597, peptide IFIGNVNNSGLK, peptide INLFDTPLETQYVR, peptide TPLPLAGPPR, peptide EGQEQEGEEMAEYR, peptide SELLVDQYLPLTK, or combinations thereof.

In an embodiment, the nutrient is α-HICA and the α-HICA is provided in an amount from about 125 mg to about 625 mg per 100 g nutritional composition.

In an embodiment, the nutrient is citrulline and the citrulline is provided in an amount from about 62 mg to about 315 mg per 100 g nutritional composition.

In an embodiment, the nutrient is a branched chain fatty acid and the branched chain fatty acid is provided in an amount from about 6.25 mg to about 12.5 mg per 100 g nutritional composition.

In another embodiment, a method for treating an individual having, or at risk of having, a medical condition is provided. The method includes providing a nutritional composition comprising milk fat globule membrane (“MFGM”), and administering the nutritional composition to the individual, wherein the medical condition is selected from the group consisting of low-grade inflammation, loss of lean body mass, skeletal muscle cell membrane instability, joint inflammation, or combinations thereof.

In an embodiment, the individual is an elderly individual.

In an embodiment, the MFGM originates from a milk source selected from the group consisting of bovine, buffalo, horse, goat, human, or combinations thereof.

In an embodiment, the composition further includes at least one nutrient selected from the group consisting of whey protein micelles, alpha-hyroxyisocaproic acid (“α-HICA”), citrulline, branched chain fatty acids, or combinations thereof.

In an embodiment, the nutritional composition further includes α-HICA and the nutritional composition is administered in an amount sufficient to provide from about 2 g to about 10 g of the α-HICA per day.

In an embodiment, the nutritional composition further includes citrulline and the nutritional composition is administered in an amount sufficient to provide from about 1 g to about 5 g of the citrulline per day.

In an embodiment, the nutritional composition further includes a branched chain fatty acid and the nutritional composition is administered in an amount sufficient to provide from about 100 mg to about 1,500 mg of the branched chain fatty acid per day.

An advantage of the present disclosure is to provide improved nutritional compositions.

Another advantage of the present disclosure is to provide nutritional compositions having beneficial anabolic nutrients.

Still yet another advantage of the present disclosure is to provide nutritional compositions that stimulate protein synthesis in humans.

Yet another advantage of the present disclosure is to provide nutritional compositions that promote muscle growth.

Still yet another advantage of the present disclosure is to provide nutritional compositions that preserve lean body mass.

Another advantage of the present disclosure is to provide nutritional compositions that mask off-flavors of nutrients in the nutritional composition.

Yet another advantage of the present disclosure is to provide nutritional compositions that has acceptable organoleptic properties.

Still yet another advantage of the present disclosure is to provide methods for administering a nutritional composition.

Additional features and advantages are described herein, and will be apparent from the following Detailed Description and the figures.

### BRIEF DESCRIPTION OF THE FIGURES

FIG. 1 shows a highly schematic structure of a whey protein micelle in accordance with an embodiment of the present disclosure.

FIG. 2 shows a Temperature Gradient-gel Electrophoresis of human milk from one mother, 7 days postpartum. Bacterial DNA (Bands 1 and 2) appearing in particular in samples 5 and 6 (the cream fractions containing MFGM).

FIG. 3 shows a Temperature Gradient-gel Electrophoresis of human milk from two mothers. Bacterial DNA (Bands indicated by the middle 2 arrows) appearing in particular in samples 2 and 3 (the cream fractions containing MFGM).

FIG. 4 shows electron micrographs of MFGM showing bacterial structures alone or in chains (marked with arrows), associate with the MFGM.

FIG. 5 shows a model of mucosal cell interactions.

FIG. 6 shows an epithelial cell response to LPS. Probiotics and MFGM lower epithelial cell responsiveness to endotoxin challenge (LPS) and related inflammatory reaction. A cumulative effect could be observed with the combination of probiotics and MFGM, suggesting a synergy between the two ingredients.

FIG. 7 shows T-cell activation. MFGM and probiotics promote T lymphocyte activation. A synergy could be observed between MFGM and B. lactis.

FIG. 8 shows B-cell activation. MFGM promotes B lymphocyte activation. Probiotics alone are less effective. A synergy could be observed between MFGM and probiotics.

### DETAILED DESCRIPTION

In this specification, the following terms have the meaning assigned to them below:

All dosage ranges contained within this application are intended to include all numbers, whole or fractions, contained within said range.

As used in this disclosure and the appended claims, the singular forms “a,” “an” and “the” include plural referents unless the context clearly dictates otherwise. Thus, for example, reference to “a polypeptide” includes a mixture of two or more polypeptides, and the like.

As used herein, “about” is understood to refer to numbers in a range of numerals. Moreover, all numerical ranges herein should be understood to include all integer, whole or fractions, within the range.

As used herein the term “amino acid” is understood to include one or more amino acids. The amino acid can be, for example, alanine, arginine, asparagine, aspartate, citrulline, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine, valine, or combinations thereof.

As used herein, “animal” includes, but is not limited to, mammals, which include but is not limited to, rodents, aquatic mammals, domestic animals such as dogs and cats, farm animals such as sheep, pigs, cows and horses, and humans. Wherein the terms “animal” or “mammal” or their plurals are used, it is contemplated that it also applies to any animals that are capable of the effect exhibited or intended to be exhibited by the context of the passage.

As used herein, the term “antioxidant” is understood to include any one or more of various substances such as beta-carotene (a vitamin A precursor), vitamin C, vitamin E, and selenium) that inhibit oxidation or reactions promoted by Reactive Oxygen Species (“ROS”) and other radical and non-radical species. Additionally, antioxidants are molecules capable of slowing or preventing the oxidation of other molecules. Non-limiting examples of antioxidants include astaxanthin, carotenoids, coenzyme Q10 (“CoQ10”), flavonoids, glutathione, Goji (wolfberry), hesperidin, lactowolfberry, lignan, lutein, lycopene, polyphenols, selenium, vitamin A, vitamin C, vitamin E, zeaxanthin, or combinations thereof.

As used herein, “complete nutrition” includes nutritional products and compositions that contain sufficient types and levels of macronutrients (protein, fats and carbohydrates) and micronutrients to be sufficient to be a sole source of nutrition for the animal to which it is being administered to. Patients can receive 100% of their nutritional requirements from such complete nutritional compositions.

As used herein, “effective amount” is an amount that prevents a deficiency, treats a disease or medical condition in an individual or, more generally, reduces symptoms, manages progression of the diseases or provides a nutritional, physiological, or medical benefit to the individual. A treatment can be patient- or doctor-related.

As used herein, “elderly” individuals include individuals that are 65 years of age or older.

As used herein, “food grade micro-organisms” means micro- organisms that are used and generally regarded as safe for use in food.

As used herein, “incomplete nutrition” includes nutritional products or compositions that do not contain sufficient levels of macronutrients (protein, fats and carbohydrates) or micronutrients to be sufficient to be a sole source of nutrition for the animal to which it is being administered to. Partial or incomplete nutritional compositions can be used as a nutritional supplement.

While the terms “individual” and “patient” are often used herein to refer to a human, the present disclosure is not so limited. Accordingly, the terms “individual” and “patient” refer to any animal, mammal or human having or at risk for a medical condition that can benefit from the treatment.

As used herein, “individual in need” means any infant, baby, child, adolescent or adult having particular physiological needs in regard to the physio-pathological conditions considered and for which the present disclosure offers an improved or alternative solution. This includes patients of all age suffering from such physio-pathological conditions.

As used herein, “long term administrations” are preferably continuous administrations for more than 6 weeks. Alternatively, “short term administrations,” as used herein, are continuous administrations for less than 6 weeks.

As used herein, “mammal” includes, but is not limited to, rodents, aquatic mammals, domestic animals such as dogs and cats, farm animals such as sheep, pigs, cows and horses, and humans. Wherein the term “mammal” is used, it is contemplated that it also applies to other animals that are capable of the effect exhibited or intended to be exhibited by the mammal.

The term “microorganism” is meant to include the bacterium, yeast and/or fungi, a cell growth medium with the microorganism, or a cell growth medium in which microorganism was cultivated.

As used herein, “milk fat globule membrane” (“MFGM”) means fatty fractions of milk (in particular cow milk or human milk) as described and defined in K. Dewettinck et al., “Nutritional and technological aspects of milk fat globule membrane material,” International Dairy Journal, vol. 18, pp. 436-457 (2008). In short, the term encompasses the membrane and membrane-associated materials that surround fat globules in mammalian milk, all and together with the other components of MFGM, referred to as “MFGM components.” For ease of language in the present document, the terms “MFGM” and “MFGM components” are used interchangeably.

As used herein, the term “minerals” is understood to include boron, calcium, chromium, copper, iodine, iron, magnesium, manganese, molybdenum, nickel, phosphorus, potassium, selenium, silicon, tin, vanadium, zinc, or combinations thereof.

As used herein, a “non-replicating” microorganism means that no viable cells and/or colony forming units can be detected by classical plating methods. Such classical plating methods are summarized in the microbiology book: James Monroe Jay, et al., “Modern food microbiology,” 7th edition, Springer Science, New York, N.Y. p. 790 (2005). Typically, the absence of viable cells can be shown as follows: no visible colony on agar plates or no increasing turbidity in liquid growth medium after inoculation with different concentrations of bacterial preparations (‘non replicating’ samples) and incubation under appropriate conditions (aerobic and/or anaerobic atmosphere for at least 24 h). For example, bifidobacteria such as Bifidobacterium longum, Bifidobacterium lactis and Bifidobacterium breve or lactobacilli, such as Lactobacillus paracasei or Lactobacillus rhamnosus, may be rendered non-replicating by heat treatment, in particular low temperature/long time heat treatment.

As used herein, a “nucleotide” is understood to be a subunit of deoxyribonucleic acid (“DNA”), ribonucleic acid (“RNA”), polymeric RNA, polymeric DNA, or combinations thereof. It is an organic compound made up of a nitrogenous base, a phosphate molecule, and a sugar molecule (deoxyribose in DNA and ribose in RNA). Individual nucleotide monomers (single units) are linked together to form polymers, or long chains. Exogenous nucleotides are specifically provided by dietary supplementation. The exogenous nucleotide can be in a monomeric form such as, for example, 5′-Adenosine Monophosphate (“5′-AMP”), 5′-Guanosine Monophosphate (“5′-GMP”), 5′-Cytosine Monophosphate (“5′-CMP”), 5+-Uracil Monophosphate (“5′-UMP”), 5′-Inosine Monophosphate (“5′-IMP”), 5′-Thymine Monophosphate (“5′-TMP”), or combinations thereof. The exogenous nucleotide can also be in a polymeric form such as, for example, an intact RNA. There can be multiple sources of the polymeric form such as, for example, yeast RNA.

“Nutritional products,” or “nutritional compositions,” as used herein, are understood to include any number of optional additional ingredients, including conventional food additives (synthetic or natural), for example one or more acidulants, additional thickeners, buffers or agents for pH adjustment, chelating agents, colorants, emulsifies, excipient, flavor agent, mineral, osmotic agents, a pharmaceutically acceptable carrier, preservatives, stabilizers, sugar, sweeteners, texturizers, and/or vitamins. The optional ingredients can be added in any suitable amount. The nutritional products or compositions may be a source of complete nutrition or may be a source of incomplete nutrition.

As used herein, sources of ω-3 fatty acids include, for example, fish oil, krill, plant sources of ω-3, flaxseed, walnut, and algae. Examples of ω-3 fatty acids include, for example, α-linolenic acid (“ALA”), docosahexaenoic acid (“DHA”), eicosapentaenoic acid (“EPA”), or combinations thereof.

As used herein, “phytochemicals” or “phytonutrients” are non-nutritive compounds that are found in many foods. Phytochemicals are functional foods that have health benefits beyond basic nutrition, are health promoting compounds that come from plant sources, and may be natural or purified. “Phytochemicals” and “Phytonutrients” refers to any chemical produced by a plant that imparts one or more health benefit on the user. Non-limiting examples of phytochemicals and phytonutrients include those that are:

i) phenolic compounds which include monophenols (such as, for example, apiole, carnosol, carvacrol, dillapiole, rosemarinol); flavonoids (polyphenols) including flavonols (such as, for example, quercetin, fingerol, kaempferol, myricetin, rutin, isorhamnetin), flavanones (such as, for example, fesperidin, naringenin, silybin, eriodictyol), flavones (such as, for example, apigenin, tangeritin, luteolin), flavan-3-ols (such as, for example, catechins, (+)-catechin, (+)-gallocatechin, (−)-epicatechin, (−)-epigallocatechin, (−)-epigallocatechin gallate (EGCG), (−)-epicatechin 3-gallate, theaflavin, theaflavin-3-gallate, theaflavin-3′-gallate, theaflavin-3,3′-digallate, thearubigins), anthocyanins (flavonals) and anthocyanidins (such as, for example, pelargonidin, peonidin, cyanidin, delphinidin, malvidin, petunidin), isoflavones (phytoestrogens) (such as, for example, daidzein (formononetin), genistein (biochanin A), glycitein), dihydroflavonols, chalcones, coumestans (phytoestrogens), and Coumestrol; Phenolic acids (such as: Ellagic acid, Gallic acid, Tannic acid, Vanillin, curcumin); hydroxycinnamic acids (such as, for example, caffeic acid, chlorogenic acid, cinnamic acid, ferulic acid, coumarin); lignans (phytoestrogens), silymarin, secoisolariciresinol, pinoresinol and lariciresinol); tyrosol esters (such as, for example, tyrosol, hydroxytyrosol, oleocanthal, oleuropein); stilbenoids (such as, for example, resveratrol, pterostilbene, piceatannol) and punicalagins;

ii) terpenes (isoprenoids) which include carotenoids (tetraterpenoids) including carotenes (such as, for example, α-carotene, β-carotene, γ-carotene, δ-carotene, lycopene, neurosporene, phytofluene, phytoene), and xanthophylls (such as, for example, canthaxanthin, cryptoxanthin, aeaxanthin, astaxanthin, lutein, rubixanthin); monoterpenes (such as, for example, limonene, perillyl alcohol); saponins; lipids including: phytosterols (such as, for example, campesterol, beta sitosterol, gamma sitosterol, stigmasterol), tocopherols (vitamin E), and omega-3, 6, and 9 fatty acids (such as, for example, gamma-linolenic acid); triterpenoid (such as, for example, oleanolic acid, ursolic acid, betulinic acid, moronic acid);

iii) betalains which include Betacyanins (such as: betanin, isobetanin, probetanin, neobetanin); and betaxanthins (non glycosidic versions) (such as, for example, indicaxanthin, and vulgaxanthin);

iv) organosulfides, which include, for example, dithiolthiones (isothiocyanates) (such as, for example, sulphoraphane); and thiosulphonates (allium compounds) (such as, for example, allyl methyl trisulfide, and diallyl sulfide), indoles, glucosinolates, which include, for example, indole-3-carbinol; sulforaphane; 3,3′-diindolylmethane; sinigrin; allicin; alliin; allyl isothiocyanate; piperine; syn-propanethial-S-oxide;

v) protein inhibitors, which include, for example, protease inhibitors;

vi) other organic acids which include oxalic acid, phytic acid (inositol hexaphosphate); tartaric acid; and anacardic acid; or

vii) combinations thereof.

As used herein, a “prebiotic” is a food substance that selectively promotes the growth of beneficial bacteria or inhibits the growth or mucosal adhesion of pathogenic bacteria in the intestines. They are not inactivated in the stomach and/or upper intestine or absorbed in the gastrointestinal tract of the person ingesting them, but they are fermented by the gastrointestinal microflora and/or by probiotics. Prebiotics are, for example, defined by Glenn Gibson et al., “Dietary Modulation of the Human Colonic Microbiota: Introducing the Concept of Prebiotics,” J. Nutr., 125: 1401-1412 (1995). Non-limiting examples of prebiotics include acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, or their hydrolysates, or combinations thereof.

As used herein, probiotic micro-organisms (hereinafter “probiotics”) are food-grade microorganisms (alive, including semi-viable or weakened, and/or non-replicating), metabolites, microbial cell preparations or components of microbial cells that could confer health benefits on the host when administered in adequate amounts, more specifically, that beneficially affect a host by improving its intestinal microbial balance, leading to effects on the health or well-being of the host. See, Salminen S., et al., “Probiotics: how should they be defined?,” Trends Food Sci. Technol., 10, 107-10 (1999). In general, it is believed that these micro-organisms inhibit or influence the growth and/or metabolism of pathogenic bacteria in the intestinal tract. The probiotics may also activate the immune function of the host. For this reason, there have been many different approaches to include probiotics into food products. Non-limiting examples of probiotics include Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, or combinations thereof.

The terms “protein,” “peptide,” “oligopeptides” or “polypeptide,” as used herein, are understood to refer to any composition that includes, a single amino acids (monomers), two or more amino acids joined together by a peptide bond (dipeptide, tripeptide, or polypeptide), collagen, precursor, homolog, analog, mimetic, salt, prodrug, metabolite, or fragment thereof or combinations thereof. For the sake of clarity, the use of any of the above terms is interchangeable unless otherwise specified. It will be appreciated that polypeptides (or peptides or proteins or oligopeptides) often contain amino acids other than the 20 amino acids commonly referred to as the 20 naturally occurring amino acids, and that many amino acids, including the terminal amino acids, may be modified in a given polypeptide, either by natural processes such as glycosylation and other post-translational modifications, or by chemical modification techniques which are well known in the art. Among the known modifications which may be present in polypeptides of the present disclosure include, but are not limited to, acetylation, acylation, ADP-ribosylation, amidation, covalent attachment of a flavanoid or a heme moiety, covalent attachment of a polynucleotide or polynucleotide derivative, covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, gamma-carboxylation, glycation, glycosylation, glycosylphosphatidyl inositol (“GPI”) membrane anchor formation, hydroxylation, iodination, methylation, myristoylation, oxidation, proteolytic processing, phosphorylation, prenylation, racemization, selenoylation, sulfation, transfer-RNA mediated addition of amino acids to polypeptides such as arginylation, and ubiquitination. The term “protein” also includes “artificial proteins” which refers to linear or non-linear polypeptides, consisting of alternating repeats of a peptide.

Non-limiting examples of proteins include dairy based proteins, plant based proteins, animal based proteins and artificial proteins. Dairy based proteins may be selected from the group consisting of casein, caseinates, casein hydrolysate, whey, whey hydrolysates, whey concentrates, whey isolates, milk protein concentrate, milk protein isolate, or combinations thereof. Plant based proteins include, for example, soy protein (e.g., all forms including concentrate and isolate), pea protein (e.g., all forms including concentrate and isolate), canola protein (e.g., all forms including concentrate and isolate), other plant proteins that commercially are wheat and fractionated wheat proteins, corn and it fractions including zein, rice, oat, potato, peanut, and any proteins derived from beans, buckwheat, lentils, pulses, single cell proteins, or combinations thereof. Animal based proteins may be selected from the group consisting of beef, poultry, fish, lamb, seafood, or combinations thereof.

As used herein, a “synbiotic” is a supplement that contains both a prebiotic and a probiotic that work together to improve the microflora of the intestine.

As used herein, the terms “treatment,” “treat” and “to alleviate” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The term does not necessarily imply that a subject is treated until total recovery. The terms “treatment” and “treat” also refer to the maintenance and/or promotion of health in an individual not suffering from a disease but who may be susceptible to the development of an unhealthy condition, such as nitrogen imbalance or muscle loss. The terms “treatment,” “treat” and “to alleviate” are also intended to include the potentiation or otherwise enhancement of one or more primary prophylactic or therapeutic measure. The terms “treatment,” “treat” and “to alleviate” are further intended to include the dietary management of a disease or condition or the dietary management for prophylaxis or prevention a disease or condition.

As used herein, a “tube feed” is a complete or incomplete nutritional product or composition that is administered to an animal's gastrointestinal system, other than through oral administration, including but not limited to a nasogastric tube, orogastric tube, gastric tube, jejunostomy tube (“J-tube”), percutaneous endoscopic gastrostomy (“PEG”), port, such as a chest wall port that provides access to the stomach, jejunum and other suitable access ports.

As used herein the term “vitamin” is understood to include any of various fat-soluble or water-soluble organic substances (non-limiting examples include vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, K1 and K2 (i.e. MK-4, MK-7), folic acid and biotin) essential in minute amounts for normal growth and activity of the body and obtained naturally from plant and animal foods or synthetically made, pro-vitamins, derivatives, analogs.

The present disclosure is related to nutritional compositions having a combination of milk-derived, bioactive lipids and anabolic nutrients to modulate low-grade inflammation, loss of lean body mass, skeletal muscle cell membrane instability, joint inflammation and poor joint health of patients including, for example, the elderly. The present disclosure is also related to methods for treating a medical condition of an individual in need of same. The medical condition may be, for example, low-grade inflammation, loss of lean body mass, skeletal muscle cell membrane instability, joint inflammation, poor joint health, or combinations thereof, or related conditions. The conditions may be due, in part, to the aging of the individual. The skilled artisan will appreciate, however, that the present nutritional compositions and methods need not be administered to an elderly individual and may be administered to any individual in need of same.

Existing nutrition support solutions for elderly and patients that have medical conditions similar to those mentioned above are lacking in effectiveness and/or appealing organoleptic properties that ensure consumer compliance. As a result, elderly individuals may not be receiving proper nutrition to combat medical conditions typically related to the aging process, and may experience, for example, significant lean body mass loss leading to loss of independence, functionality and quality of life. Elderly individuals may also experience a decline in cognitive ability, and the healthcare costs associated with these morbidities are high.

One known nutrient that can be used to combat medical conditions typically associated with aging includes certain lipid bioactives such as, for example, ω-3 fatty acids. Examples of ω-3 fatty acids include docosahexaenoic acid (“DHA”), eicosapentaenoic acid (“EPA”) and α-linolenic acid (“ALA”). EPA, an ω-3 polyunsaturated fatty acid, has been shown to attenuate skeletal muscle atrophy in cancer cachexia as well as sepsis and to reduce unloading-induced bone loss through a common cellular signaling pathway by minimizing activation of nuclear factor-κβ (“NF-κβ”). EPA can impact musculoskeletal health through both an attenuated loss of lean body mass and bone mineral density through targeted inhibition of NFkβ. Further, EPA can enhance skeletal muscle protein synthesis (as mediated through the mTOR pathway) and reduce endogenous muscle proteolysis (as mediated through the ubiquitin-proteasome pathway), respectively, under catabolic, disuse or aging conditions. Nutritional compositions having similar ω-3 fatty acids can result in preserved lean body mass, which can provide tonic loading to the underlying bone and act as an osteogenic stimulus for bone turnover to minimize fracture risk.

Typical ω-3 fatty acids are limited in their use, however, by poor organoleptic properties. In this manner, ω-3 fatty acids do not have desirable tastes or odors and may be less enticing to consumers. If the consumer does not like the taste or odor of a composition including ω-3 fatty acids, it will be more difficult for the consumer to maintain compliance with a diet including same.

Applicants have surprisingly found that milk fat globule membrane components (“MFGM”) can provide several health benefits to elderly individuals, or other individuals suffering from, or at risk for, conditions including, for example, low-grade inflammation, immobilization/disability, loss of lean body mass, skeletal muscle cell membrane instability, cognitive decline, join inflammation, and poor joint health. The skilled artisan will appreciate that, while these conditions are typically associated with the elderly, individuals of any age can present with similar conditions.

More specifically, Applicants have found that MFGM can modulate low-grade inflammation typically associated with aging. Low-grade inflammation can be especially problematic for elderly individuals because the inflammation can increase the anabolic threshold for skeletal muscle protein synthesis and, thus, increase the dose amounts needed for lean body mass production. The attenuated low-grade inflammation can also minimize the endogenous skeletal muscle proteolysis and create synergies with anabolic nutrients by lowering the threshold needed to allow for stimulation of skeletal muscle protein synthesis via the anabolic nutrients. The reduction of inflammation with MFGM may also help to diminish joint pain as associated with aging, rheumatoid arthritis, etc.

Administration of MFGM is also beneficial because MFGM may be selectively incorporated in skeletal muscle and provide enhanced skeletal muscle cell membrane stability. The enhanced skeletal muscle cell membrane stability provided by MFGM allows the proper functioning of integrin-mediated signaling. Integrins are transmembrane proteins that link the extracellular membrane to the cell cytoskeleton and, therefore, mediate the transduction of mechanical forces (i.e., resistance exercise) into chemical signals. The loss of integrin-mediated signaling can lead to a progressive loss of muscle function due to a failure to maintain normal sarcomeric cytoarchitecture (i.e., muscle contraction). See, Developmental Biology 338(1): pp. 15-27 (2010). Indeed, the combination of nutrition plus exercise is a critical stimulus for increasing skeletal muscle protein synthesis, improving lean body mass and enhancing functional mobility in the elderly population.

The benefits of administration of MFGM to an individual may also be enhanced when the MFGM is administered in combination with other functional anabolic nutrients. For example, branched chain amino acids, such as leucine, can act as signaling molecules to stimulate muscle protein synthesis. Further, the amino acid arginine provides many beneficial effects in the body including, for example, modulation of immune function, wound healing, hormone secretion, vascular tone, insulin sensitivity, and endothelial function. The skilled artisan will appreciate that the present nutritional compositions are not limited to the use of leucine and arginine and that other functional nutrients may also be used in combination with MFGM.

As mentioned briefly above, however, many nutrients that are used in nutritional compositions to provide a specific nutritional benefit to a consumer impart an undesirable taste or odor to the composition making it unappealing for consumption. As a result, the desired biological result is not achieved when the consumer refuses to ingest the composition due to its poor organoleptic properties. Therefore, the anabolic nutrients that may be administered with the MFGM of the present disclosure should be delivered in a palatable way to provide tolerable physical and organoleptic properties and to increase patient compliance.

Leucine is a branched chain amino acid that provides beneficial effects in the body. Branched chain amino acids (“BCAA”) are indispensable, or essential, amino acids, which means that the BCAAs must be provided exogenously to allow for muscle protein synthesis. Once consumed, BCAAs, especially leucine, can serve as signaling molecules to stimulate muscle protein synthesis. This signaling can be employed via two mechanisms. The first mechanism is stimulation of insulin release since leucine is a strong secretagogue. The second mechanism is more direct as leucine can stimulate the eukaryotic inducing factor that turns on muscle protein synthesis. As a dietary supplement, leucine has also been found to slow the degradation of muscle tissue by increasing the synthesis of muscle proteins in aged rats.

It is possible and may be desirable, however, to provide all three BCAAs (i.e., leucine, isoleucine, and valine) in a nutritional composition since the large increase of one BCAA can cause a relative deficiency of the other two BCAAs. As BCAAs are known for their undesirable sensory profile, addition of analogs such as α-HICA as well as designer, or high quality, proteins such as, for example, whey protein micelles is a effective way of delivering the benefit while improving patient compliance and therefore clinical outcome leading to better quality of life as well as health economic advantages. Further, combinations with immunomodulating agents such as lactowolfberry can bring synergistic benefits to the patient with low graded inflammation, suppressed anabolism and immunosenescence (e.g., elderly, or those with, or at risk of, illness).

Whey protein is one of the most abundant natural sources of the branched-chain amino acids (e.g., leucine, isoleucine and valine). Because the nutritional profile of whey protein is among the best sources for such amino acids it is very desirable for use in nutritional compositions. Indeed, the combination of these three essential amino acids makes up approximately ⅓ of skeletal muscle in the human body, and plays an important role in protein synthesis. Branched-chain amino acids may also be used to aid in the recovery of burn victims, as well as for supplementation for strength athletes.

More specifically, whey protein is among the richest natural sources of leucine (12-15% by weight of the total amino acids), including about 1 g of leucine per 10 g of whey protein micelles in the whey protein. However, the amount of leucine necessary to significantly improve protein synthesis in humans is reported to be approximately 3 g or more delivered in a bolus serving. As a result, it is necessary to provide more than 30 g of whey protein to achieve 3 g of leucine. However, the flavor of leucine is typically unpleasant when included in doses that are efficacious in the stimulation of protein synthesis in humans. Indeed, the sensory properties of leucine include a bitter mouth taste that is unpleasant to consumers.

As such, oral nutritional products have been limited in their ability to deliver efficacious amounts of branch chain amino acids because of the flavor profile. For example, prior art beverages are either limited by the inclusion of whey protein, which provides unacceptable viscosity, or leucine, which provides unacceptable organoleptics. In addition, whey protein has the habit of gellification when heated in neutral pH conditions. Therefore, the beverage applications for branch chain amino acids are extremely limited. Further, tablet and pill delivery of branch chain amino acids is also not convenient as a result of the dose to be administered (3 or more grams at a time).

Applicants have surprisingly found that it is possible to combine whey protein micelles with the free amino acid leucine, or other branched chain amino acids, to create compositions (e.g., a beverage) for the purpose of supporting muscle growth. Specifically, nutritional compositions of the present disclosure may include whey protein micelles and a significant amount of leucine, but do not have bitter or off-flavors that are typically associated with doses of leucine that are efficacious in the stimulation of protein synthesis in humans. Applicants have surprisingly found, therefore, that whey protein micelles can be utilized as a mask to offset the bitterness of off-flavor amino acids in beverages and other oral nutritional products. Although the present disclosure refers to the use of whey protein micelles and leucine, the skilled artisan will immediately appreciate that other branch chain amino acids, or amino acids, may also be employed in similar uses.

Without wishing to be bound to any theory, it is believed that the structure of the protein micelles and their interaction with the leucine (or other off-flavor nutrients) prevents the unpleasant bitterness perception by the consumer. As such, whey protein micelles can act as a masking substance for preventing the unpleasant bitterness perception of a specific nutrient by masking a bitter taste receptor present at the surface of the tongue. As presented by the Noriao Ishibashi model, bitterness is an unpleasant gustative sensory perception that often induces food rejection. Sensitivity to bitterness varies from 1 to 500 as a function of each specific person. See, Ishibashi, N. et al., “A Mechanism for Bitter Taste Sensibility in Peptides,” Agr. Biol. Chem. 52, 819-827 (1988).

Whey protein micelles are spherical (regular shape close to natural casein micelles), mono-dispersed micro-gels obtained by auto assembling of native whey proteins during heat treatment at a very specific pH. Whey protein micelles have unique characteristics and properties including, for example, a narrow size distribution with a diameter between 100 and 900 nm and a polydispersity index below 0.2, a turbidity value measured at 500 nm (between 20 and 50 absorbance units for a 4% protein solution) that is stable for 10 minutes, and a spherical shape as imaged by TEM microscopy.

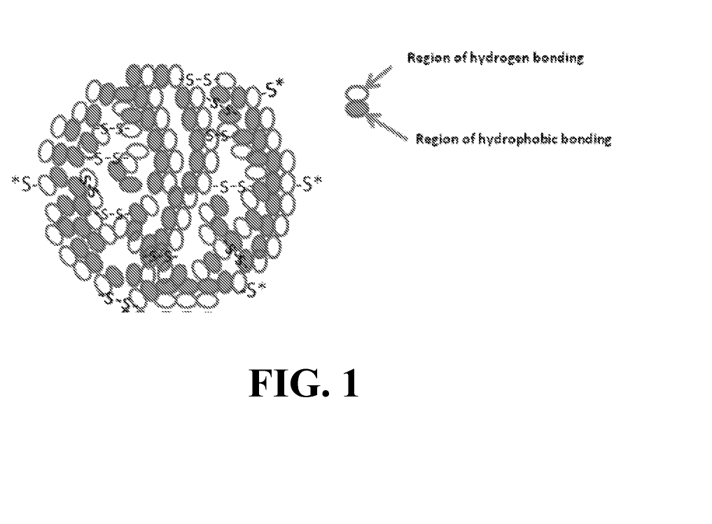

FIG. 1 illustrates a schematic representation of the micelles that may be used in the present disclosure, wherein the whey proteins are arranged in such a way that the hydrophilic parts of the proteins are oriented towards the outer part of the agglomerate and the hydrophobic parts of the proteins are oriented towards the inner “core” of the micelle. The name “whey protein micelle” is indicative of homology with casein micelles based on the following criteria: shape, size, and whitening properties, but also the whey protein micelle is a spherical whey protein micro-gel of denatured whey protein. Both physical and chemical interactions are involved in whey protein microgels or whey protein micelle. In FIG. 1, S* indicates accessible thiol/activated thiol from cysteine, and S—S indicates disulfide bridges stabilizing the whey protein micelle. This energetically favorable configuration offers good stability to these structures in a hydrophilic environment. As such, the micelles consist essentially of spherical agglomerates of denatured whey protein. The micelles are particularly characterised by their regular, spherical shape.

Due to their dual character (hydrophilic and hydrophobic), this denatured state of the protein seems to allow interaction with a hydrophobic phase, e.g., a fat droplet or air, and a hydrophilic phase. The whey protein micelles, therefore, have perfect emulsifying and foaming properties.

The micelles may have an extremely sharp size distribution such that more than 80% of the micelles produced will have a size smaller than 1 micron, preferably between 100 nm and 900 nm, more preferably between 100-770 nm, most preferably between 200 and 400 nm. Without wishing to be bound by theory, it is thought that during micelle formation, the micelles reach a “maximum” size, due to the overall electrostatic charge of the micelles repelling any additional protein molecule, such that the micelles cannot grow in size any longer. This accounts for the narrow size distribution.

The whey protein micelles of the present disclosure may be produced by the methods described in International Application PCT/EP2007/052877, filed Mar. 26, 2007; International Application PCT/EP2007/052900, filed Mar. 27, 2007; and U.S. Ser. No. 12/280,244, filed Aug. 21, 2008, the entire contents of each of which are included herein by reference. An advantage of using the methods described in these applications is that the whey protein micelles prepared accordingly have not been submitted to any mechanical stress leading to reduction of the particle size during formation, contrary to conventional processes known in the art. Instead, the methods induce spontaneous micellization of whey proteins during heat treatment in the absence of shearing. The skilled artisan will appreciate, however, that the micelles may be produced by methods other than those described in the above-mentioned applications.

Any commercially available whey protein isolates or concentrates may be used to obtain the whey protein micelles. For example, whey protein obtained by any process for the preparation of whey protein known in the art, as well as whey protein fractions prepared therefrom or proteins such as β-lactoglobulin, α-lactalbumin and serum albumin. In particular, sweet whey obtained as a by-product in cheese manufacture, acid whey obtained as a by-product in acid casein manufacture, native whey obtained by milk microfiltration or rennet whey obtained as a byproduct in rennet casein manufacture may be used as the whey protein. The whey protein may be from a single source or from mixtures of any sources. However, the skilled artisan will appreciate that the present disclosure is not restricted to whey isolates from bovine origin, but pertains to whey isolates from all mammalian animal species, such as from sheep, goats, horses, and camels.

Other health benefits provided by whey proteins include enhancement of muscle development and building, as well as muscle maintenance in children, adults or elderly people, enhancement of the immune function, improvement of cognitive function, control of blood glucose such that they are suitable for diabetics, weight management and satiety, anti-inflammatory effects, wound healing and skin repair, lowering of the blood pressure, etc.

Accordingly, Applicants have found that whey protein micelles may be used to mask the poor flavor profile of branched chain amino acids such as leucine, isoleucine and valine. In this manner, nutritional compositions of the present disclosure may provide functional nutrients to an individual while also being desirable for consumption by the individual.

In another embodiment, α-hydroxycaproic acid (“HICA”) may be used as a palatable substitute to leucine. α-HICA (a.k.a., leucic acid) is a product of leucine metabolism and occurs naturally in many fermented protein products (e.g., cheese, wine, soy sauce, etc.). α-HICA may be beneficial for individuals because it can aid in maximizing the anabolism and minimizing the catabolism of muscle tissue.

Specifically, Applicants have found that α-HICA can deliver superior benefits due to taste profile as well as complementary metabolic benefits. For example, α-HICA is a leucine metabolite with anabolic benefits directly related to protein synthesis. Since α-HICA is a leucine metabolite, it will provide many of the same benefits described above with respect to leucine. However, unlike leucine, α-HICA does not suffer from the same unpleasant bitterness perception by the consumer and, therefore, serves as a palatable substitute to leucine.

In an embodiment, α-HICA may be provided in an amount from about 125 mg to about 625 mg per 100 g of the nutritional composition assuming an embodiment wherein the nutritional composition includes 1,600 grams and is a complete daily feeding for an adult. α-HICA may also be provided in an amount from about 200 mg to about 500 mg per 100 g of the nutritional composition, or about 300 mg per 100 g of the nutritional composition. In another embodiment, the nutritional compositions may include α-HICA in sufficient amounts to provide from about 2 g to about 10 g of α-HICA per day, or from about 4 g to about 8 g, or about 6 g.

Similar to the BCAAs, other amino acids can provide metabolic benefits. For example, arginine is an alpha amino acid that is classified as a semiessential or conditionally essential amino acid, depending on the developmental stage and health status of the individual. Arginine has many effects in the body that include, among others, modulation of immune function, would healing, hormone secretion, vascular tone, insulin sensitivity, and endothelial function. As such, arginine is useful as a functional anabolic nutrient in nutritional compositions for the elderly or individuals in need of such benefits.

Arginine is metabolized into citrulline and nitric oxide (“NO”) via the enzyme nitric oxide synthase (“NOS”). However, only a portion of the arginine consumed by an individual remains available for metabolization to NO. As much as 60% of ingested arginine is metabolized in the liver by arginase before entering the circulation, where any remaining arginine may be metabolized to citrulline and NO. Accordingly, the ingestion of a large quantity of an arginine-rich dietary supplement is required in order to provide an effective amount of arginine to an individual in need of same. This limits the usefulness of arginine for nutritional compositions. Additionally, and similar to leucine, arginine has somewhat unappealing organoleptic properties.

An alternative source for arginine is the endogenous production of arginine from the amino acid citrulline. This route contributes about 20% to whole body arginine production. Citrulline is a precursor to L-arginine and is produced in the intestine. Just as arginine is converted to citrulline and NO, L-citrulline is converted to arginine in the mitochondria via a part of the urea cycle. The majority of circulating L-citrulline is converted in the kidneys, which are compromised of highly metabolically active tissue. As such, L-citrulline circulating in the bloodstream is first converted to arginine and then in cells to citrulline and NO. Further, citrulline enters circulation without being metabolized by the liver, with almost complete conversion to arginine in the kidneys. Therefore, smaller amounts of citrulline are required to provide the body with effective amounts of arginine in vivo. Moreover, ingestion of citrulline, or a precursor of citrulline, therefore, is able to provide many of the same benefits as ingestion of arginine including, for example, modulation of immune function, would healing, hormone secretion, vascular tone, insulin sensitivity, and endothelial function, but with lesser amounts.

Significantly, the conversion of L-citrulline to arginine occurs continuously, as long as L-citrulline is circulating in the bloodstream. As a result, circulating L-citrulline makes it possible to maintain elevated concentrations of arginine over time. Accordingly, the administration of L-citrulline may be used as a substitute for arginine to overcome arginine deficiencies including, for example, its poor organoleptic properties.

Further, macrophage cells can directly convert citrulline to arginine and maintain the ability of macrophages to kill invading cells. Indeed, it is thought that there is a prolonged production of nitric oxide by the conversion of citrulline to arginine by macrophage. This, in turn, can reduce the level of inflammation in an individual and the detrimental effects of infection on anabolism. One benefit of reduced inflammation is increased insulin sensitivity which leads to increased anabolism.

Guadagni and Biolo indicate that additional protein may be needed in individuals with inflammation (such as the elderly or individuals with illness) in part to maintain the levels of arginine and glutamine. See, Guadagni and Biolo, “Effects of inflammation and/or inactivity on the need for dietary protein,” Curr. Opin. Clin. Nutr. Metab. Care, 12(6):617-22 (November 2009). Citrulline can serve to maintain arginine levels. Additionally, it can help to maintain glutamine levels since glutamine conversion to citrulline in the small intestine will be reduced by a feedback signal from the citrulline provided exogenously. This will reduce the need for muscle catabolism to provide arginine and glutamine for bodily functions.

It is further possible that citrulline can improve the maintenance of lean body mass in elderly that do a limited amount of exercise and/or physical therapy. Citrulline has been shown to have an anabolic effect in malnourished aged animals. The anabolic signal in the elderly population is typically down-regulated. The addition of citrulline will provide a strong boost to this signal. This improved recovery from physical activity will allow for accelerated recovery from inactivity due to aging. Further, improved preservation of lean body mass will help to maintain metabolic homeostasis and functional mobility, and the preservation of bone mass density can reduce the risk of fracture thus leading to improved quality of life as well as healthcare cost savings. A reduction in cost of care could also be realized based on reduced number physical therapy sessions and a faster return to full independent living and a return to work.

As such, Applicants have found that citrulline can be provided in nutritional compositions to provide advantageous effects in the body, as well as improved organoleptic properties when compared to arginine. In an embodiment, citrulline may be provided in an amount from about 62 mg to about 315 mg per 100 g of the nutritional composition assuming an embodiment wherein the nutritional composition includes 1,600 grams and is a complete daily feeding for an adult. Citrulline may also be provided in an amount from about 100 mg to about 200 mg per 100 g of the nutritional composition, or about 250 mg per 100 g of the nutritional composition. In another embodiment, the nutritional compositions may include citrulline in sufficient amounts to provide from about 0.5 g to about 10 g of citrulline per day, or from about 1 g to about 5 g, or about 3 g.

The nutritional compositions of the present claims may include, therefore, MFGM alone, or in combination with anabolic nutrients that provide additional functional effects in the body. These ingredient combinations can provide the advantages described above, as well as bone and joint health benefits through direct effect on skeletal muscle cell membrane (e.g., MFGM, branched chain fatty acids) or indirect (e.g., enhancing of lean body mass and subsequent strength can increase mobility thus leading to increase of bone mineral density, cognitive health, functionality and quality of life, etc).

The present disclosure may provide nutritional compositions including MFGM in an amount of from about 0.01 g to about 20 g per 100 g of nutritional composition. In an embodiment, the nutritional compositions include from about 1 g to about 15 g per 100 g of the nutritional composition, or from about 5 g to about 10 g per 100 g of the nutritional composition. In another embodiment, the nutritional compositions include MFGM in an amount between about 0.1% and about 20% by weight of total proteins of the nutritional composition. In an embodiment, the nutritional compositions include MFGM in an amount between about 1.0% and about 15% by weight of total proteins of the nutritional composition, or between about 5% and about 10% by weight of total proteins of the nutritional composition.

The MFGM of the present nutritional compositions may originate from a source selected from the group consisting of butter milk, butter milk fractions, defatted butter milk, delactosylated buttermilk, buttermilk fraction obtained by microfiltration, or ultrafiltration, fractions recovered from whey protein concentrate, sweet whey, acid whey, whey cream or fat associated fraction from whey containing phospholipids. In an embodiment, MFGM originates from a milk source selected from the group consisting of bovine, buffalo, horse, goat, human milk, or combinations thereof.

The human milk fat globule membrane protein composition is still largely unknown, although it counts for 2-4% of the total milk protein content. See, Stephania Quaranta et al., “Human proteome enhancement: High-recovery method and improved two dimensional map of colostral fat globule membrane proteins,” Electrophoresis, 22, pp. 1810-1818 (2001). The present disclosure includes an amount of human MFGM ranging from 0.1% to 20% of the total protein of the composition.

In mammalian milk, the fat phase generally accounts for around 40 g/L and is mainly composed of triglycerides (96% of total fat), diglycerides (2% of total fat), and complex lipids (1% of total fat). Triglycerides synthesized in the smooth endoplasmic reticulum of the mammary alveolar cell coalesce into large droplets which migrate to the apical plasma membrane of the cell. The lipid droplets then push against and progressively become enveloped in the membrane of the mammary gland epithelial cells. These membranes, budded off around the milk lipids as they are being secreted by the cells, are named the milk fat globule membranes. The milk fat globule membrane contains specific glycoproteins such as lactoferrin, mucins, lactadherin and xanthine oxidase as well as complex polar lipids such as glycerophospholipids and sphingolipids. Many of these components are present in human milk in much higher concentrations than in bovine milk.

MFGM may further include a component selected from the group consisting of sphingomyelin, phosphatidyl ethanolamine, phosphatidylcholine, phosphatidyl inositol, phosphatidyl serine, cholesterol, gangliosides, mucin1, xantine-oxidase/dehydrogenase, periodicacid schiff, CD36, butyrophilin, adipophilin, PAS 6/7, fatty-acid binding protein, lactoferrin, lactaldherin, peptide ETTVFENLPEK, peptide SFQLFGSPPGQR, peptide GSNFQLDQLQGR, peptide FQFIQVAGR597, peptide IFIGNVNNSGLK, peptide INLFDTPLETQYVR, peptide TPLPLAGPPR, peptide EGQEQEGEEMAEYR, peptide SELLVDQYLPLTK, or combinations thereof.

A variety of ingredients enriched in MFGM are commercially available. MFGM can be present in cream, buttermilk and whole milk. For product development use, buttermilk fraction is the source most often used due to its relatively high concentration of MFGM components. Examples of commercially available buttermilk products include buttermilk (product code 26048) from Land O'Lakes, Inc. MN, buttermilk protein concentrate fractions from Fonterra Cooperative Group (Auckland, New Zealand); a phospholipid-rich fraction derived from MFGM from the Fonterra Cooperative Group Ltd. (Auckland, New Zealand); buttermilk from Büllinger Butterei (Bullingen, Belgium), Foster Farms Dairy (Modesto, Calif.), Dairy America Inc. (Fresno, Calif.), Dairy Farmers of America (Kansas City, Mo.); First Milk Ingredients Limited, Paisley, UK and Laban Up products of Gulf & Safa Dairies, United Arab Emirates.

However, whey protein fractions enriched in MFGM are also commercially available. Examples include LACPRODAN® MFGM-10, a whey protein fraction enriched in MFGM produced by Arla Food Ingredients amba, Denmark. Other examples of material may include Promilk 602 E from Ingredia Lacto prosperité AG (Ingredia SA, Arras France), and from any suppliers containing MFGM (at least 1.5% of MFGM components in order to be able to adjust the amount of MFGM to total protein).

Depending on the whey protein/casein ratio (50/50, 70/30) in the nutritional compositions, one source of MFGM casein based or milk based such as butter milk or Promilk 602E can be mixed in respect with this ratio with a whey protein. Further, the MFGM can comprise proteins, gangliosides, phospholipids, or combinations thereof.

In an embodiment, the nutritional compositions of the present disclosure include at least one probiotic. The first interaction of probiotics with the host occurs at the level of the gut mucosa. Probiotics have been widely demonstrated to protect the host against infections and potentially improve specific disease outcomes. Among the key criteria for probiotic strain selection is their capacity to adhere to the intestinal mucosa. This appears to be a prerequisite for blocking pathogen entry and modulating protective immune functions.

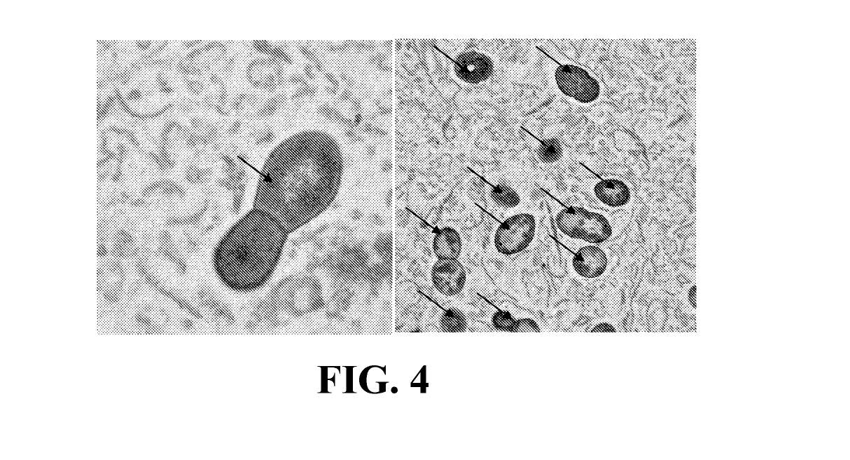

In studies examining the interaction of bovine milk fat globule membrane with intestinal epithelial cells (IEC) in vitro, Applicants disrupted the MFGM. Although bacteria were not directly detected in fractions of intact membranes, the disruption of the membranes by ultra-sound resulted in contamination of the IEC cultures. The bacteria appeared to come from the MFGM and not from the IEC cultures. In support of this, electron micrographs of bovine MFGM reveal the presence of bacterial cocci in chains (see, FIG. 4). Without wishing to be bound by theory, Applicants believe that bacteria binding to, or encapsulation of bacteria and/or their components within, the milk fat globule membrane may facilitate transport of the microbial components through the gastrointestinal system, ensure their delivery to appropriate sites in the mucosal tissues of the suckling neonate and, together with other factors in the milk fat globule membrane, modulate immunological processes.

It is hypothesized that the association of MFGM and probiotics leads to potentiation (synergy) of the beneficial effects seen using probiotics or MFGM alone. Mechanistically, it can be hypothesized, for example, that MFGM is a significant source of lipids to the consumer and that certain bacteria may increase the expression of molecules involved in lipid absorption. It follows that it should be possible to produce similar beneficial effects in consumers of the present nutritional compositions by supplementing the nutritional compositions with probiotics and MFGM. The probiotics can be pre-blended with MFGM before addition to the composition or the two preparations (probiotics, MFGM) can be added separately to the nutritional compositions of the present disclosure.

The probiotic may be present in the nutritional compositions in an amount equivalent to between 10³ and 10¹² cfu/g of dry composition. The bacteria may be used live, inactivated or dead or even be present as fragments such as DNA or cell wall materials. In other words, the quantity of bacteria which the formula contains is expressed in terms of the equivalent colony forming units of bacteria irrespective of whether they are, all or partly, live, inactivated, dead or fragmented Preferably, the probiotic is present in an amount equivalent to between 10⁷ to 10¹² cfu/g of dry composition.

The probiotic bacterial strain may be any lactic acid bacteria or Bifidobacteria with established probiotic characteristics. The probiotic of the present disclosure may be any probiotic bacteria or probiotic microorganism that have been or can be originated, found, extracted or isolated in milk upon excretion, preferably in human breast milk. Suitable probiotic lactic acid bacteria include Lactobacillus rhamnosus ATCC 53103 obtainable inter alia from Valio Oy of Finland under the trademark LGG, Lactobacillus rhamnosus CGMCC 1.3724, Lactobacillus reuteri ATCC 55730 and Lactobacillus reuteri DSM 17938 obtainable from Biogaia, Lactobacillus fermentum VRI 003 and Lactobacillus paracasei CNCM I-2116, Lactobacillus johnsonii CNCM I-1225, Lactobacillus helveticus CNCM I-4095, Bifidobacterium breve CNCM I-3865, Bifidobacterium longum CNCM I-2618.

Suitable probiotic Bifidobacteria strains include Bifidobacterium longum ATCC BAA-999 sold by Morinaga Milk Industry Co. Ltd. of Japan under the trademark BB536, the strain of Bifidobacterium breve sold by Danisco under the trademark Bb-03, the strain of Bifidobacterium breve sold by Morinaga under the trademark M-16V and the strain of Bifidobacterium breve sold by Institut Rosell (Lallemand) under the trademark R0070. A particularly preferred Bifidobacterium strain is Bifidobacterium lactis CNCM I-3446 which may be obtained from the Christian Hansen Company of Denmark under the trademark Bb12. A mixture of suitable probiotic lactic acid bacteria and Bifidobacteria may be used.

The MFGM and the probiotic of the present disclosure can interact together at the biological level. In particular, the MFGM can enhance or promote the biological effect of the probiotic. In one embodiment, the MFGM enables the probiotic to have a biological effect that it would otherwise not have in absence of MFGM. In an embodiment, the MFGM and probiotics could have a synergistic effect in that the beneficial biological effect of the probiotic will be increased. Such biological effect can comprise the effect on the maturation of the immune system and/or of the gut, the promotion of the anti-infection effect and/or the reduction of inflammation.

In an embodiment, the nutritional compositions of the present disclosure include proteins that are able to bind to a probiotic surface. As such, nutritional compositions of the present disclosure may contain a protein source in an amount of not more than 3.7 or 2.0 g/100 kcal, or 1.8 to 2.0 g/100 kcal for infants or up to 10 g/100 kcal for adults. In an embodiment, the nutritional compositions are formulated for an adult and include a protein source in an amount from about 2 to 8 g/100 kcal, or from about 4 to 6 g/100 kcal, or about 5 g/100 kcal. The source and type of protein (e.g., from whey, casein, or combinations thereof) in the composition of the present disclosure (i.e., the protein content not originating from MFGM) is not believed to be critical to the present disclosure provided that the minimum requirements for essential amino acid content are met and provided that satisfactory growth is ensured. However, in one embodiment it is preferred that more than 50% or more than 60% by weight of the protein source is whey (hence insuring a best balanced amino-acid profile). Thus, protein sources based on whey, casein, or combinations thereof may be used as well as protein sources based on soy. As far as whey proteins are concerned, the protein source may be based on acid whey, sweet whey, or combinations thereof, and may include alpha-lactalbumin and beta-lactoglobulin in desired proportions.

In an embodiment, the protein source is based on modified sweet whey. Sweet whey is a readily available by-product of cheese making and is frequently used in the manufacture of infant formulas based on cows' milk. However, sweet whey includes a component that is undesirably rich in threonine and poor in tryptophan called caseino-glyco-macropeptide (“CGMP”). Removal of the CGMP from sweet whey results in a protein with a threonine content closer to that of human milk. This modified sweet whey can then be supplemented with those amino acids in respect of which it has a low content (principally histidine and tryptophan). A process for removing CGMP from sweet whey is described in EP 880902. Using modified sweet whey as the principal protein in the protein source enables all essential amino acids to be provided at a protein content between 1.8 and 2.0 g/100 kcal. Such protein sources have been shown in animal and human studies to have a protein efficiency ratio, nitrogen digestibility, biological value and net protein utilization comparable to standard whey-adapted protein sources with a much higher protein content per 100 kcal and to result in satisfactory growth despite their reduced protein content. If modified sweet whey is used as the protein source, it is preferably supplemented by free histidine in an amount of from 0.1 to 1.5% by weight of the protein source.

The proteins may be intact, hydrolyzed, or combinations thereof. It may be desirable to supply partially hydrolyzed proteins (degree of hydrolysis between 2% and 20%), for example for individuals believed to be at risk of developing cows' milk allergy. If hydrolyzed proteins are required, the hydrolysis process may be carried out as desired and as is known in the art.

For example, a whey protein hydrolysate may be prepared by enzymatically hydrolyzing the whey fraction in one or more steps. For an extensively hydrolyzed protein, the whey proteins may be subjected to triple hydrolysis using Alcalase 2.4 L (EC 940459), then Neutrase 0.5 L (obtainable from Novo Nordisk Ferment AG) and then pancreatin at 55° C. Alternatively, for a less hydrolyzed protein, the whey may be subjected to a two-stage hydrolysis using trypsin, chymotrypsin, pancreatin, or combinations thereof, as described in EP 322589. If the whey fraction used as the starting material is substantially lactose free, it is found that the protein suffers much less lysine blockage during the hydrolysis process. This enables the extent of lysine blockage to be reduced from about 15% by weight of total lysine to less than about 10% by weight of lysine; for example about 7% by weight of lysine which greatly improves the nutritional quality of the protein source. In one embodiment of the present disclosure, the MFGM preparation is subjected to the same proteolytic treatment.

Nutritional compositions of the present disclosure may contain a carbohydrate source. Any carbohydrate source conventionally found in nutritional compositions such as lactose, saccharose, maltodextrin, starch and mixtures thereof may be used although the preferred source of carbohydrates is lactose. In an embodiment, the carbohydrate source contributes between 35% and 60% of the total energy of the nutritional compositions.

Nutritional compositions of the present disclosure may also contain a source of lipids, beside the lipids from the MFGM components. The lipid source may be any lipid or fat which is suitable for use in nutritional compositions. Sources of fat includes, but are not limited to, high oleic sunflower oil and high oleic safflower oil. The essential fatty acids linoleic and α-linolenic acid may also be added as may small amounts of oils containing high quantities of preformed arachidonic acid and docosahexaenoic acid such as fish oils or microbial oils. In total, the fat content is preferably such as to contribute between about 10% and about 10% of the total energy of the nutritional compositions.

In an embodiment, the nutritional compositions further include a source of ω-3 fatty acids. The source of ω-3 fatty acids may be selected from the group consisting of fish oil, krill, plant sources containing ω-3 fatty acids, flaxseed, walnut, algae, or combinations thereof. The ω-3 fatty acids may be selected from the group consisting of α-linolenic acid (“ALA”), docosahexaenoic acid (“DHA”), eicosapentaenoic acid (“EPA”), or combinations thereof.

In an embodiment, the nutritional compositions further include at least one nucleotide selected from the group consisting of a subunit of deoxyribonucleic acid (“DNA”), a subunit of ribonucleic acid (“RNA”), polymeric forms of DNA and RNA, yeast RNA, or combinations thereof. In an embodiment, the at least one nucleotide is an exogenous nucleotide.

In an embodiment, the nutritional compositions further include a phytonutrient selected from the group consisting of flavanoids, allied phenolic compounds, polyphenolic compounds, terpenoids, alkaloids, sulphur-containing compounds, or combinations thereof. The phytonutrient may be selected from the group consisting of carotenoids, plant sterols, quercetin, curcumin, limonin, or combinations thereof.

In an embodiment, the nutritional compositions further include a source of protein, as discussed above. The source of protein may be selected from the group consisting of dairy based proteins, plant based proteins, animal based proteins, artificial proteins, or combinations thereof. The dairy based proteins may be casein, caseinates, casein hydrolysate, whey, whey hydrolysates, whey concentrates, whey isolates, milk protein concentrate, milk protein isolate, or combinations thereof. The plant based proteins may be soy protein, pea protein, canola protein, wheat and fractionated wheat proteins, corn proteins, zein proteins, rice proteins, oat proteins, potato proteins, peanut proteins, green pea powder, green bean powder, spirulina, proteins derived from vegetables, beans, buckwheat, lentils, pulses, single cell proteins, or combinations thereof. In an embodiment, the protein source contributes between 15% and 35% of the total energy of the nutritional compositions.

In an embodiment, the nutritional compositions further include a probiotic, as discussed above, the probiotic selected from the group consisting of Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, non-replicating microorganisms, or combinations thereof.

In an embodiment, the nutritional compositions further include an amino acid selected from the group consisting of alanine, arginine, asparagine, aspartate, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine, valine, or combinations thereof.

In an embodiment, the nutritional compositions further include an antioxidant selected from the group consisting of astaxanthin, carotenoids, coenzyme Q10 (“CoQ10”), flavonoids, glutathione, Goji (wolfberry), hesperidin, lactowolfberry, lignan, lutein, lycopene, polyphenols, selenium, vitamin A, vitamin C, vitamin E, zeaxanthin, or combinations thereof.

In an embodiment, the nutritional compositions further include a vitamin selected from the group consisting of vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, K1 and K2 (i.e., MK-4, MK-7), folic acid, biotin, or combinations thereof.

In an embodiment, the nutritional compositions further include a mineral selected from the group consisting of boron, calcium, chromium, copper, iodine, iron, magnesium, manganese, molybdenum, nickel, phosphorus, potassium, selenium, silicon, tin, vanadium, zinc, or combinations thereof. Minerals may be added in salt form. The presence and amounts of specific minerals and other vitamins will vary depending on the intended population.

In an embodiment, the nutritional composition includes branched chain fatty acids that are present in the nutritional composition in an amount from about 6.25 mg to about 12.5 mg/100 g nutritional composition and assuming the nutritional composition includes 1,600 grams and is a complete feeding for a day for an adult. Alternatively, the nutritional compositions may be provided in an amount to provide from about 100 mg to about 1,500 mg of branched chain fatty acids per day. Alternatively, the nutritional compositions may include from about 0.5% to about 5% branched chain fatty acids by weight of total fatty acids.

In an embodiment, the nutritional compositions also include a prebiotic. In an embodiment, the prebiotic is selected from the group consisting of acacia gum, alpha glucan, arabinogalactans, beta glucan, dextrans, fructooligosaccharides (“FOS”), fucosyllactose, galactooligosaccharides (“GOS”), galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, their hydrolysates, or combinations thereof. A combination of prebiotics may be used such as 90% GOS with 10% short chain FOS such as the product sold under the trademark Beneo P95, or 10% inulin such as the product sold under the trademark Beneo® HP, ST or HSI.

An example of a useful prebiotic is a mixture of galacto-oligosaccharide(s), N-acetylated oligosaccharide(s) and sialylated oligosaccharide(s) in which the N-acetylated oligosaccharide(s) includes 0.5 to 4.0% of the oligosaccharide mixture, the galacto-oligosaccharide(s) includes 92.0 to 98.5% of the oligosaccharide mixture and the sialylated oligosaccharide(s) includes 1.0 to 4.0% of the oligosaccharide mixture. This mixture is hereinafter referred to as “the preferred prebiotic mixture.” In an embodiment, the nutritional compositions of the present disclosure contain from 2.5 to 15.0 wt % of the preferred prebiotic mixture on a dry matter basis with the proviso that the composition comprises at least 0.02 wt % of an N-acetylated oligosaccharide, at least 2.0 wt % of a galacto-oligosaccharide and at least 0.04 wt % of a sialylated oligosaccharide.

Suitable N-acetylated oligosaccharides include GalNAcα1,3Galβ1,4Glc and Galβ1,6GalNAcα1,3Galβ1,4Glc. The N-acetylated oligosaccharides may be prepared by the action of glucosaminidase and/or galactosaminidase on N-acetyl-glucose and/or N-acetyl galactose. Equally, N-acetyl-galactosyl transferases and/or N-acetyl-glycosyl transferases may be used for this purpose. The N-acetylated oligosaccharides may also be produced by fermentation technology using respective enzymes (recombinant or natural) and/or microbial fermentation. In the latter case, the microbes may either express their natural enzymes and substrates or may be engineered to produce respective substrates and enzymes. Single microbial cultures or mixed cultures may be used. N-acetylated oligosaccharide formation can be initiated by acceptor substrates starting from any degree of polymerisation (DP) from DP=1 onwards. Another option is the chemical conversion of keto-hexoses (e.g. fructose) either free or bound to an oligosaccharide (e.g., lactulose) into N-acetylhexosamine or an N-acetylhexosamine containing oligosaccharide as described in Wrodnigg, T. M, et al., Angew. Chem. Int. Ed. 38:827-828 (1999).

Suitable galacto-oligosaccharides include Galβ1,6Gal, Galβ1,6Galβ1,4Glc Galβ1,6Galβ1,6Glc, Galβ1,3Galβ1,3Glc, Galβ1,3Galβ1,4Glc, Galβ1,6Galβ1,6Galβ1,4Glc, Galβ1,6Galβ1,3Galβ1,4Glc Galβ1,3Galβ1,6Galβ1,4Glc, Galβ1,3Galβ1,3Galβ1,4Glc, Galβ1,4Galβ1,4Glc and Galβ1,4Galβ1,4Galβ1,4Glc. Synthesised galacto-oligosaccharides such as Galβ1,6Galβ1,4Glc Galβ1,6Galβ1,6Glc, Galβ1,3Galβ1,4Glc, Galβ1,6Galβ1,6Galβ1,4Glc, Galβ1,6Galβ1,3Galβ1,4Glc and Galβ1,3Galβ1,6Galβ1,4Glc, Galβ1,4Galβ1,4Glc and Galβ1,4Galβ1,4Galβ1,4Glc and mixtures thereof are commercially available under the trade marks Vivinal® and Elix'or®. Other suppliers of oligosaccharides are Dextra Laboratories, Sigma-Aldrich Chemie GmbH, and Kyowa Hakko Kogyo Co., Ltd. Alternatively, specific glycosyltransferases, such as galactosyltransferases may be used to produce neutral oligosaccharides.

Suitable sialylated oligosaccharides include NeuAca2,3Galβ1,4Glc and NeuAca2,6Galβ1,4Glc. These sialylated oligosaccharides may be isolated by chromatographic or filtration technology from a natural source such as animal milks. Alternatively, they may also be produced by biotechnology using specific sialyltransferases either by enzyme based fermentation technology (recombinant or natural enzymes) or by microbial fermentation technology. In the latter case microbes may either express their natural enzymes and substrates or may be engineered to produce respective substrates and enzymes. Single microbial cultures or mixed cultures may be used. Sialyl-oligosaccharide formation can be initiated by acceptor substrates starting from any degree of polymerization (DP) from DP=1 onwards.

In an embodiment, the nutritional compositions may contain emulsifiers and stabilizers such as soy lecithin, citric acid esters of mono- and di-glycerides, and the like. The nutritional compositions may also optionally contain other substances which may have a beneficial effect such as lactoferrin, nucleotides, nucleosides, and the like.

In an embodiment, the nutritional compositions are in a form selected from the group consisting of tablets, capsules, liquids, chewables, soft gels, sachets, powders, syrups, liquid suspensions, emulsions, solutions, or combinations thereof.

In an embodiment, the nutritional compositions are oral nutritional supplements. Alternatively, the nutritional compositions may be tube feedings. The nutritional compositions may also be a source of complete nutrition. Alternatively, the nutritional compositions may be a source of incomplete nutrition.

The present nutritional compositions may be administered in one large bolus, or in several feedings per day. A full day of feeding of the nutritional compositions of the present disclosure for an adult may be from about 1000 kcal to about 2000 kcal. In an embodiment, a full day feeding of the present nutritional compositions for an adult is about 1500 kcal. As such, at 1.0 kcal/mL, the present nutritional compositions may be administered to an adult in an amount of about 1500 mL per day. The skilled artisan will appreciate, however, that the present nutritional compositions may be administered according to feeding regimens that are tailored to meet the specific needs of the individuals consuming the compositions.

The nutritional compositions may be prepared in any suitable manner. For example, a nutritional composition may be prepared by blending together the protein source, the carbohydrate source, and the fat source in appropriate proportions. If used, the emulsifiers may be included in the blend. The vitamins and minerals may be added at this point but are usually added later to avoid thermal degradation. Any lipophilic vitamins, emulsifiers and the like may be dissolved into the fat source prior to blending. Water, preferably water which has been subjected to reverse osmosis, may then be mixed in to form a liquid mixture.

The liquid mixture may then be thermally treated to reduce undesired viable bacterial loads. For example, the liquid mixture may be rapidly heated to a temperature in the range of about 80° C. to about 110° C. for about 5 seconds to about 5 minutes. This may be carried out by steam injection or by heat exchanger; for example a plate heat exchanger.

The liquid mixture may then be cooled to about 60° C. to about 85° C., for example, by flash cooling. The liquid mixture may then be homogenized, for example, in two stages at about 7 MPa to about 40 MPa in the first stage and about 2 MPa to about 14 MPa in the second stage. The homogenized mixture may then be further cooled to add any heat sensitive components such as vitamins and minerals. The pH and solids content of the homogenized mixture is conveniently standardized at this point.

The homogenized mixture is transferred to a suitable drying apparatus such as a spray drier or freeze drier and converted to powder. The powder should have a moisture content of less than about 5% by weight.

The selected probiotic(s) may be cultured according to any suitable method and prepared for addition to the nutritional compositions by freeze-drying or spray-drying, for example. Alternatively, bacterial preparations can be bought from specialist suppliers such as Christian Hansen and Morinaga already prepared in a suitable form for addition to food products such as nutritional compositions.

The selected probiotic may be blended to any MFGM preparation before drying. This blend could be homogenized to favor association between MFGM and probiotics. After drying the blend can be used as a module (added to liquid formula) or MFGM can be mixed to formula powder.

Probiotic powder and MFGM powders could be mixed by dry mixing, forming a specific blend. This blend could be added together in the final nutritional composition powder. Alternately, the blend can be added extemporary to the liquid formula.

MFGM preparation could also be added during formula process before homogenization and pasteurization/heat treatment sterilization up to UHT treatment for a liquid formula. After drying the probiotic could be incorporated by dry mixing.

MFGM preparation could also be considered as a good natural emulsifier to reduce the emulsifiers conventionally used in nutritional compositions. In one embodiment, the probiotic and/or the MFGM preparations are added separately or together to a ready-to-drink or ready-to-dilute nutritional composition such as a powder nutritional compositions. Such addition(s) can occur during one of the last process steps of the manufacturing/packaging of the composition or can occur just before the use of the composition by the intended user. In such instance, the MFGM preparation and/or the probiotic preparation can be provided separately from the powder or liquid nutritional composition.

By way of example and not limitation, the following Examples are illustrative of embodiments of the present disclosure.

### EXAMPLES

### Example 1

The following example presents scientific data developing and supporting the concept of interactions between probiotics and MFGM in mammals breast milk. It follows that compositions comprising both MFGM and probiotics present an advantageous effect.

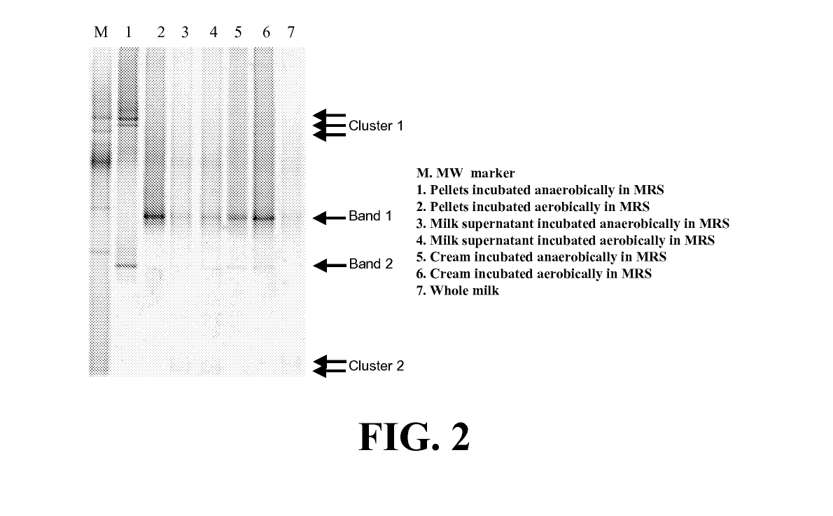

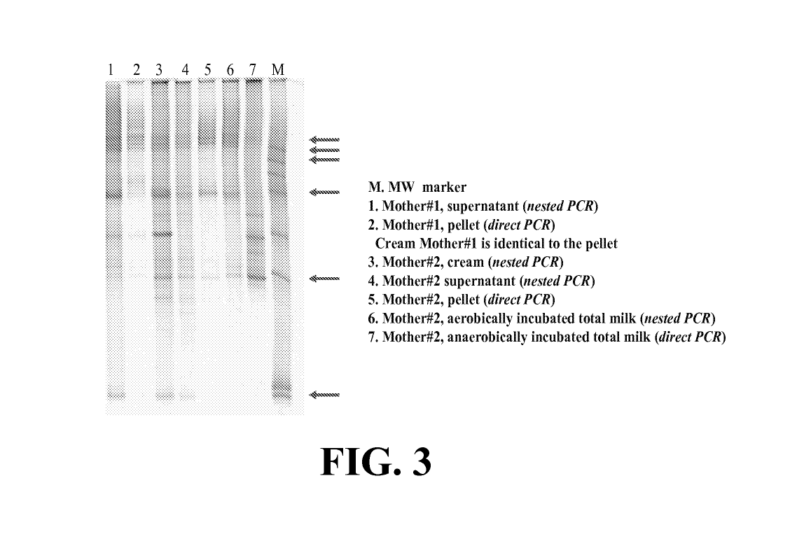

In is known from prior art that a low dose of micro-organisms and a range of microbial DNA are contained in human breast milk and are associated with the milk cellular compartment. Applicants have herewith hypothesized that bacteria in milk may be associated with other milk compartments besides the cellular component. For example, they may be transported within the MFGM or casein micelles. Applicants have analyzed the presence of microbial DNA signals in various fractions of mammals milk. In FIGS. 2 and 3, temporal Temperature Gradient-gel Electrophoresis (“TTGE”) was used. The experiments detected that strong bacterial DNA signals were also found in the cream fraction of the milk (FIGS. 2 and 3). The finding support the concept that the MFGM contains bacterial components and/or that the MFGM may bind to or “envelop” the bacteria in the milk.

Further the inventors have investigated the transportation of microbial load. FIG. 4 shows microbial component co-located with MFGM. Applicants hypothesize that bacterial binding to or encapsulation of bacteria and/or their components within the MFGM, may facilitate transport of the microbial components through the gastrointestinal system Without wishing to be bound to a theory, Applicants believe that this may enhance their delivery to appropriate sites in the mucosal tissues of the consumer. Together with other factors in the MFGM it may further modulate immunological processes.

### Example 2

Proteomics studies of the human MFGM both internally (R&D reports RDLS-RD040131 Donnet-Hughes et al; RDLS-RD040096 Warth and Donnet-Hughes) and externally, have identified several proteins which are considered to influence bacterial growth or survival, are involved in the recognition of and/or response to microbes or their components, or are known to interact with other proteins that do so. For example, Toll-like receptors (“TLRs”) are a class of cell membrane receptors that recognize structurally conserved molecules derived from microbes and activate immune responses. They are believed to play a key role in the innate immune system. The MFGM comprises molecules which are co-receptors/molecules of TLR signaling complexes as well as potential ligands for these complexes.

Differential responses to pro-inflammatory factors have been observed in vitro, suggesting that the MFGM and/or MFGM fractions may support immune defense mechanisms without promoting exaggerated responses (see Table 1).

TABLE 1
Effect of various fractions containing MFGM (whole or digest) on the
expression of factors, showing up-regulation or down-regulation.

| | | | Intestinal epithelial cells | | | | |
|---|---|---|---|---|---|---|---|
| | Peripheral blood | | | | | Tissue inhibitor | |
| | mononuclear cells | | NF-κB | Il-1ra | | of metallo- | |
| | IFN- | LPS- | acti- | production | | proteinase-1 | |
| | induced | induced | vation | Basal | IFN- | Basal | |
| | IL- | TNF- | TNF- | pro- | in- | pro- | TNF- |
| Fraction | 12p70 | α | induced | duction | duced | duction | induced |
| MFGM | ↓ | ↓ | ↑ | ↓ | — | — | — |
| MFGM | — | ↓ | ↑ | ↓ | — | ↑ | —/↑ |
| Digest | | | | | | | |
| MFGM | ↓ | ↑ | ↑ | | | | |
| MFGM | — | ↑ | ↑ | | | | |
| Digest | | | | | | | |

Taken together, it is plausible that delivery of bacteria and/or bacterial components in the presence of MFGM may influence the composition of the intestinal microbiota. It could further modulate immune responses in the recipient host such that there is tolerance to components of the normal microbiota and dietary antigens and protection against potential pathogens or danger signals.

Combinations of MFGM and/or MFGM fractions with probiotic organisms and/or components could be used to educate the immune system and provide protection against infections.

Overall, MFGM and/or MFGM fractions, when associated with probiotics and/or probiotic components, could promote the interaction of probiotics with the host and modulate downstream processes involved in defense mechanisms. Applicants believe that MFGM and/or MFGM fractions, in combination with probiotics and/or probiotic components, may help modulate the neonatal microbiota composition, support immune development and trigger efficient protective host defense reactions. This can include immune responses, against various pathogens or other environmental danger. This can be linked to capacity of the probiotics to confer optimal delivery to the host and/or promote host responsiveness to exogenous and endogenous signals.

### Example/Experimental Data 3

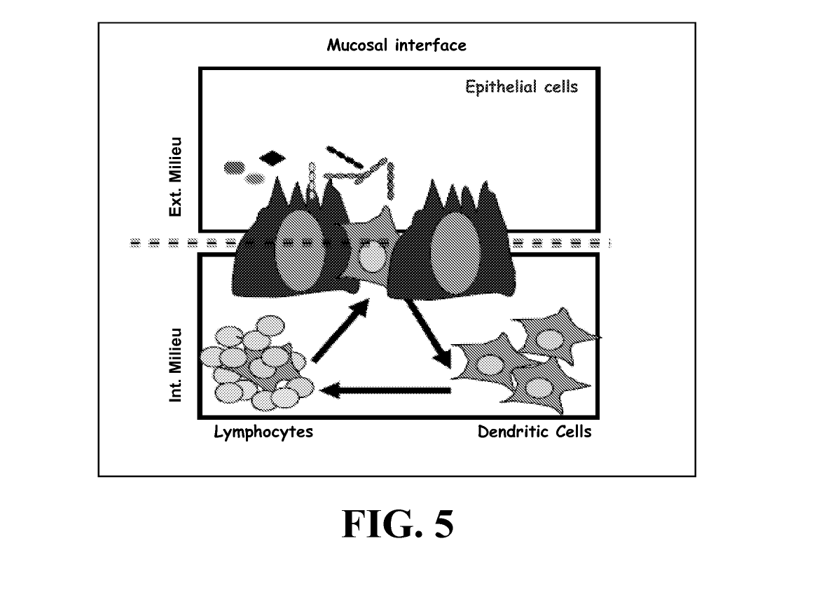

The Interactions of MFGM fractions with the host and potential synergistic effect with probiotics was evaluated in vitro in a system mimicking the gut mucosa (FIG. 5). The data support a beneficial effect of a combination of MFGM and probiotics on host defences against infections and modulation of inflammatory conditions.

HT29C134 NFkB Reporter Assays:

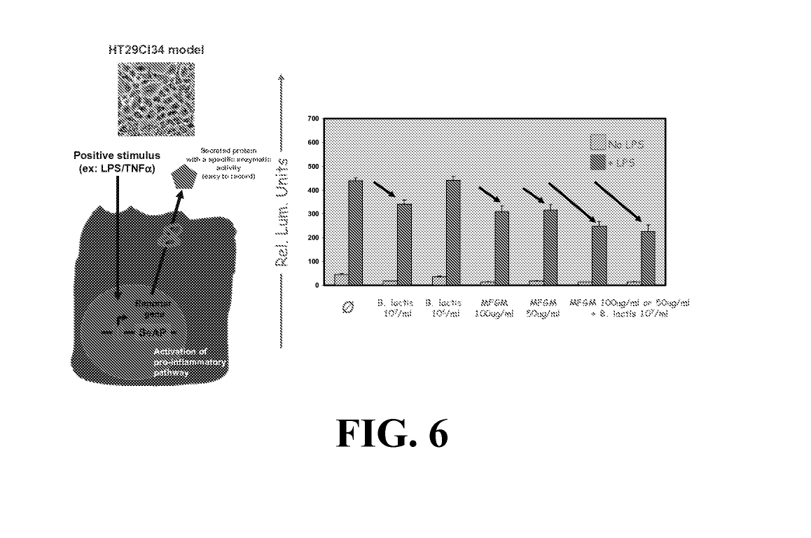

HT-29 (human colonic epithelial cells) cell line express endogenous TLRs. TLR signaling activates the NF-kB transcription factor either through MyD88 or TRIF adaptator proteins and leads to expression of inflammatory genes. HT-29 cell lines were also transfected with a reporter construct, in which the secreted alkaline phosphatase (SeAP) is expressed under the control of a NF-kB inducible promoter. Thus, a given TLR stimulation can be reported by measuring secreted alkaline phosphatase activity which reflects NF-kB activation in this so called HT-29C134 cell line developed at Nestlé research Center. To assess TLR-mediated inflammation cascade, HT-29C134 reporter gene system was used to measure levels of NF-kB activation after a pre-treatment with Bifidobacterium lactis NCC2818 (B. lactis) at 10e⁶ or 10e⁷ CFU/mL and/or MFGM preparation at 50 ug/mL or 100 ug/mL final, then a challenge or not with LPS (100 ng/mL) was done and SeAP activity assessed by a fluorometric assay and expressed in relative fluorescent units. Bifidobacterium lactis NCC2818 (B. lactis) was selected as representative of Bifidobacteria species that are important components of infant early life gut microbial colonization. FIG. 6 shows the cumulative effect of MFGM and probiotics in this model indicating a synergistic effect. It is evidenced that MFGM decreases the responsiness of epithelial cells to an endotoxin challenge. MFGM+probiotics (B. Lactis) exhibits a stronger effect than MFGM alone or probiotics alone.

B and T Cell Stimulation Assays:

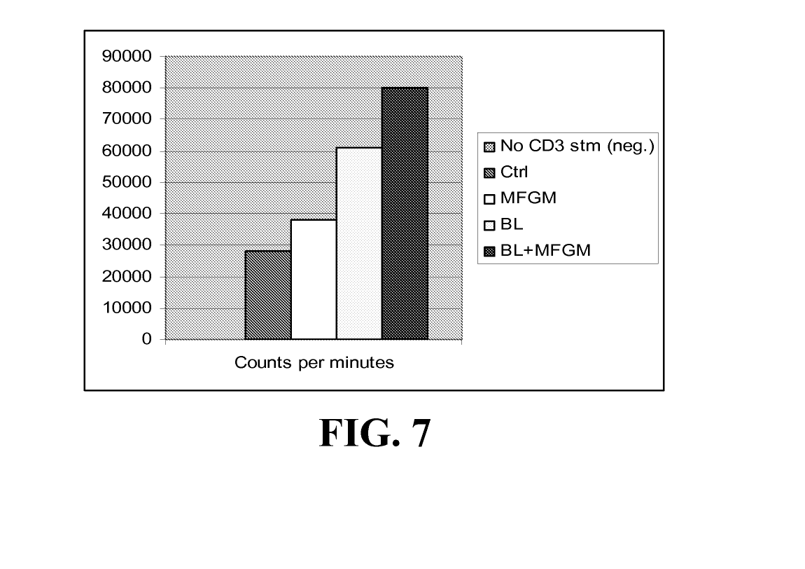

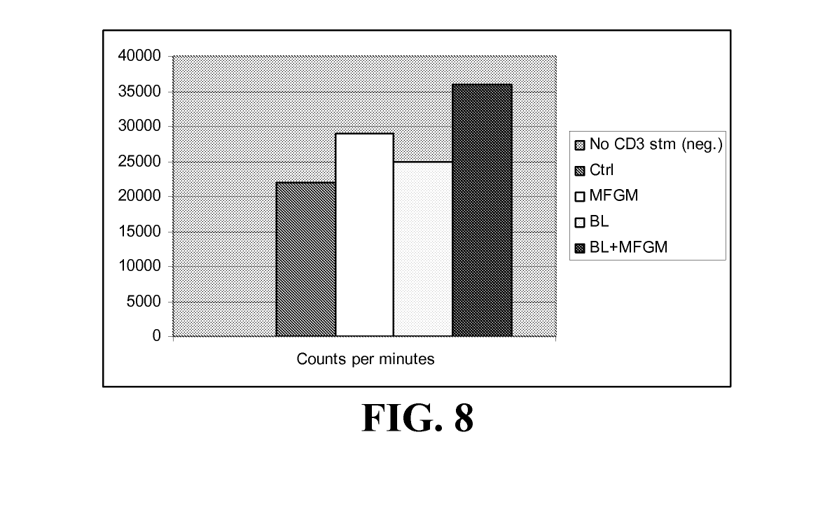

Lymphocytes suspensions were prepared from pooled mesenteric and inguinal lymph nodes isolated from 6-8 weeks old C57BL/6 mice. Cells were suspended in IMDM culture medium supplemented with 5×10−5 M β-mercaptoethanol, 1 mM glutamine, 100 U/mL penicillin, 100 μg/mL streptomycin and 10% FCS. For anti-CD3 (T-cell specific) and anti-CD40 (B-cell specific) stimulation, 96-well flat-bottom plates were coated with 50 μL PBS containing 2.5 μg/mL anti-CD3 (clone: 2C11) or 5 μg/mL anti-CD40 (clone: FGK-45) 1-3 h at 37° C. After extensive washing, serial 3-fold dilutions of MLN cell suspensions were added per well. After 3 days, 1 μCi/well ³H-thymidine was added for the last 18 hours prior to harvesting. When said NCC2818 (B. lactis) obtained from standard cultures were added to the well as well as MFGM preparation at MOI 100 and/or 100 μg/mL final respectively. Results from assay performed with optimal titration of lymphoid cells (111,000 cells/well) are presented in FIGS. 7 and 8. The results highlight the synergistic effect of a probiotic with MFGM.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A nutritional composition comprising milk fat globule membrane (“MFGM”) and at least one nutrient selected from the group consisting of whey protein micelles, alpha-hydroxyisocaproic acid (“α-HICA”), citrulline, branched chain fatty acids, and combinations thereof.

2. The nutritional composition of claim 1, wherein the whey protein micelles comprise at least one branched chain amino acid selected from the group consisting of leucine, isoleucine, valine, and combinations thereof.

3. The nutritional composition of claim 1 comprising a probiotic selected from the group consisting of Bifidobacterium lactis CNCM I-3446, Lactobacillus rhamnosus GG ATCC 53103, Lactobacillus rhamnosus CGMCC 1.3724, Bifidobacterium longum BB536 deposited under ATCC BAA-999, Lactobacillus Reuteri ATCC55730, Lactobacillus Reuteri DSM-17938, Lactobacillus paracasei CNCM I-2116, Lactobacillus johnsonii CNCM I-1225, Lactobacillus helveticus CNCM I-4095, Bifidobacterium breve CNCM I-3865, Bifidobacterium longum CNCM I-2618, and combinations thereof.

4. The nutritional composition of claim 3, wherein the MFGM comprises proteins or bioactive proteins able to bind with or biologically interact with the probiotic.

5. The nutritional composition of claim 3, wherein the MFGM comprises gangliosides or phospholipids able to bind with or biologically interact with the probiotic, the gangliosides or phospholipids being present in an amount of 0.03% to 5% by weight of total proteins.

6. The nutritional composition of claim 1, wherein said MFGM is present in an amount of about 0.1% to about 15% by weight of total proteins.

7. The nutritional composition of claim 1, wherein the MFGM is present in an amount of about 0.01 g to about 15 g of MFGM per 100 g of the nutritional composition.

8. The nutritional composition of claim 1 further comprising a prebiotic selected from the group consisting of fructo-oligosaccharides, galacto-oligosaccharides, cow milk oligosaccharides, and combinations thereof.

9. The nutritional composition of claim 1, wherein the MFGM originates from a source selected from the group consisting of butter milk, butter milk fractions, defatted butter milk, delactosylated buttermilk, buttermilk fraction obtained by microfiltration or ultrafiltration, fractions recovered from whey protein concentrate, sweet whey, acid whey, whey cream or fat associated fraction from whey containing phospholipids, and combinations thereof.

10. The nutritional composition of claim 1, wherein the MFGM comprises a component selected from the group consisting of sphingomyelin, phosphatidyl ethanolamine, phosphatidylcholine, phosphatidyl inositol, phosphatidyl serine, cholesterol, gangliosides, mucin1, xantine-oxidase/dehydrogenase, periodic acid schiff, CD36, butyrophilin, adipophilin, PAS 6/7, fatty-acid binding protein, lactoferrin, lactaldherin, peptide ETTVFENLPEK, peptide SFQLFGSPPGQR, peptide GSNFQLDQLQGR, peptide FQFIQVAGR597, peptide IFIGNVNNSGLK, peptide INLFDTPLETQYVR, peptide TPLPLAGPPR, peptide EGQEQEGEEMAEYR, peptide SELLVDQYLPLTK, and combinations thereof.

11. The nutritional composition of claim 1, wherein the nutrient is α-HICA and the α-HICA is provided in an amount from about 125 mg to about 625 mg per 100 g nutritional composition.

12. The nutritional composition of claim 1, wherein the nutrient is citrulline and the citrulline is provided in an amount of about 62 mg to about 315 mg per 100 g nutritional composition.

13. The nutritional composition of claim 1, wherein the nutrient is a branched chain fatty acid and the branched chain fatty acid is provided in an amount from of about 6.25 mg to about 12.5 mg per 100 g nutritional composition.

14. A method for treating an individual having, or at risk for having, a medical condition, the method comprising the steps of:
providing a nutritional composition comprising milk fat globule membrane (“MFGM”); and
administering the nutritional composition to the individual, wherein the medical condition is selected from the group consisting of low-grade inflammation, loss of lean body mass, skeletal muscle cell membrane instability, poor joint health, joint inflammation, immobilization/disability, cognitive decline, and combinations thereof.

15. The method of claim 14, wherein the individual is an elderly individual.

16. The method of claim 14, wherein the MFGM originates from a milk source selected from the group consisting of bovine, buffalo, horse, goat, human, and combinations thereof.

17. The method of claim 14, wherein the composition comprises at least one nutrient selected from the group consisting of whey protein micelles, alpha-hyroxyisocaproic acid (“α-HICA”), citrulline, branched chain fatty acids, and combinations thereof.

18. The method of claim 17, wherein the nutrient is α-HICA and the nutritional composition is administered in an amount sufficient to provide from about 2 g to about 10 g of α-HICA per day.

19. The method of claim 17, wherein the nutrient is citrulline and the nutritional composition is administered in an amount sufficient to provide from about 1 g to about 5 g of citrulline per day.

20. The method of claim 17, wherein the nutrient is a branched chain fatty acid and the nutritional composition is administered in an amount sufficient to provide from about 100 mg to about 1.500 mg of the branched chain fatty acid per day.
